(19) United States
(12) Patent Application Publication
(10) Pub. No.: US 2012/0107801 A1
(43) Pub. Date: May 3, 2012

(54) HIGH-EFFICIENCY HOMOLOGOUS RECOMBINATION IN THE OIL-PRODUCING ALGA, NANNOCHLOROPSIS

(76) Inventors: Oliver Kilian, Alameda, CA (US); Chris Benemann, Walnut Creek, CA (US); Bertrand Vick, Berkeley, CA (US)

(21) Appl. No.: 13/246,700

(22) Filed: Sep. 27, 2011

Related U.S. Application Data
(63) Continuation-in-part of application No. 12/581,812, filed on Oct. 19, 2009.
(60) Provisional application No. 61/386,958, filed on Sep. 27, 2010.

(51) Int. Cl. C12Q 1/68; C12N 15/79

(52) U.S. Cl. 435/6.1; 435/471; 435/320.1

(57) ABSTRACT

Transformation methods are provided for introducing deoxyribonucleic acid (DNA) into the nucleus of an algal cell. A transformation construct may be prepared, with the transformation construct having a first sequence of DNA similar to a corresponding first sequence of nuclear DNA, a second sequence of DNA similar to a corresponding second sequence of the nuclear DNA, and a sequence of DNA inserted between the first and second sequences of DNA of the transformation construct. A target sequence of DNA inserted between the first and second corresponding sequences of the nuclear DNA may be transformed, resulting result in replacement of the target sequence of DNA with the sequence of DNA of interest.

### CROSS-REFERENCE TO RELATED APPLICATIONS

The present application is a continuation in part of U.S. Non-Provisional patent application Ser. No. 12/581,812 filed on Oct. 19, 2009, titled “Homologous Recombination is an Algal Nuclear Genome,” which is hereby incorporated by reference. The present application also claims the benefit and priority of U.S. Provisional Patent Application Ser. No. 61/386,558 filed on Sep. 27, 2010, titled High-Efficiency Homologous Recombination in the Oil-Producing Alga, Nannochloropsis,” which is hereby incorporated by reference.

The present application is related to U.S. Non-Provisional patent application Ser. No. 12/480,635 filed on Jun. 8, 2009, titled “VCP-Based Vectors for Algal Cell Transformation,” which is hereby incorporated by reference.

The present application is related to U.S. Non-Provisional patent application Ser. No. 12/480,611 filed on Jun. 8, 2009, titled “Transformation of Algal Cells,” which is hereby incorporated by reference.

### REFERENCE TO SEQUENCE LISTINGS

The present application is filed with sequence listing(s) attached hereto and incorporated by reference.

### BACKGROUND OF THE INVENTION

1. Field of the Invention

This invention relates to molecular biology, and more specifically, to the expression of exogenous DNA elements in algal cells.

2. Description of Related Art

Manipulating the DNA of a cell may confer upon the cell new abilities. For example, a transformed cell (i.e., a cell that has taken-up exogenous DNA) may be more robust than the wild-type cell. For many so-called model biological systems (i.e., well-studied organisms), the DNA elements for transformation have been developed. For other organisms, of which less is known, transformation is a major milestone that must be achieved to facilitate genetic engineering. Complicating this challenge is the need for efficient, non-random transformation of these organisms. Accordingly, there is a need for homologous recombination in an algal nuclear genome.

### SUMMARY OF THE INVENTION

Provided herein are exemplary transformation methods for introducing deoxyribonucleic acid (DNA) into the nucleus of an algal cell. A transformation construct may be prepared, with the transformation construct having a first sequence of DNA similar to a corresponding first sequence of nuclear DNA, a second sequence of DNA similar to a corresponding second sequence of the nuclear DNA, and a sequence of DNA of interest inserted between the first and second sequences of DNA of the transformation construct. A target sequence of DNA inserted between the first and second corresponding sequences of the nuclear DNA may be transformed, resulting in replacement of the target sequence of DNA with the sequence of DNA of interest. In further exemplary embodiments, the sequence of DNA of interest may comprise an antibiotic resistance marker, a promoter sequence and an antibiotic resistance marker, or a gene for nutrient assimilation or biosynthesis of a metabolite. A phenotypic characteristic of the algal cell may be changed or new characteristics may be imparted to the algal cell.

Also provided is an exemplary transformation construct, the transformation construct having a first sequence of DNA similar to a corresponding first sequence of nuclear DNA of an algal cell, a second sequence of DNA similar to a corresponding second sequence of nuclear DNA of the algal cell, and a sequence of DNA of interest inserted between the first and second sequences of the transformation construct. According to a further exemplary embodiment, the sequence of DNA of interest may further comprise DNA to compromise or destroy wild-type functioning of a gene for nutrient assimilation or biosynthesis of a metabolite.

### BRIEF DESCRIPTION OF THE DRAWINGS

FIG. 1 is a diagram showing how exemplary deoxyribonucleic acid (DNA) sequences may be utilized for introducing DNA into the nucleus of an algal cell, according to one exemplary embodiment.

FIG. 2 is a flow chart showing an exemplary method for homologous recombination in an algal nuclear genome.

FIG. 3 shows an exemplary DNA sequence (SEQ. ID. NO. 1), which includes at least a portion of a nitrate reductase gene.

FIG. 4 shows an exemplary transformation construct (SEQ. ID. NO. 2), which incorporates nitrate reductase DNA sequences for the flanks of the transformation construct.

FIG. 5 is a gel showing a PCR analysis of several transformants obtained with the transformation construct illustrated in FIG. 4.

FIG. 6 shows the knock-out (“KO”) of a nitrate reductase (“NR”) gene by homologous recombination in Nannochloropsis sp. Structures of NR-KO transformation constructs (“TC”), wild-type (Wt) genes, and homologous recombination (“HR”) products are also shown.

FIG. 7 shows the knock-out (“KO”) of a nitrite reductase (“NiR”) gene by homologous recombination in Nannochloropsis sp. Structures of NiR-KO transformation constructs (“TC”), wild-type (Wt) genes, and homologous recombination (“HR”) products are also shown.

FIG. 8 shows growth of Wt, NR-KO (NR1 and NR2), and NiR-KO (NiR1 and NiR2) with different nitrogen sources, relative to Wt in 1 mM NH₄Cl.

FIG. 9 shows PCR analysis of NR-KO and NiR-KO transformants.

FIGS. 10A-10C show an exemplary DNA sequence (SEQ. ID. NO. 3), which includes at least a portion of a nitrate reductase gene.

FIGS. 11A-11B show an exemplary DNA sequence (SEQ. ID. NO. 4), which includes at least a portion of a nitrite reductase gene.

FIG. 12 shows an exemplary DNA sequence (SEQ. ID. NO. 5), which includes at least a portion of a VCP1 3′ untranslated region.

### DETAILED DESCRIPTION OF THE INVENTION

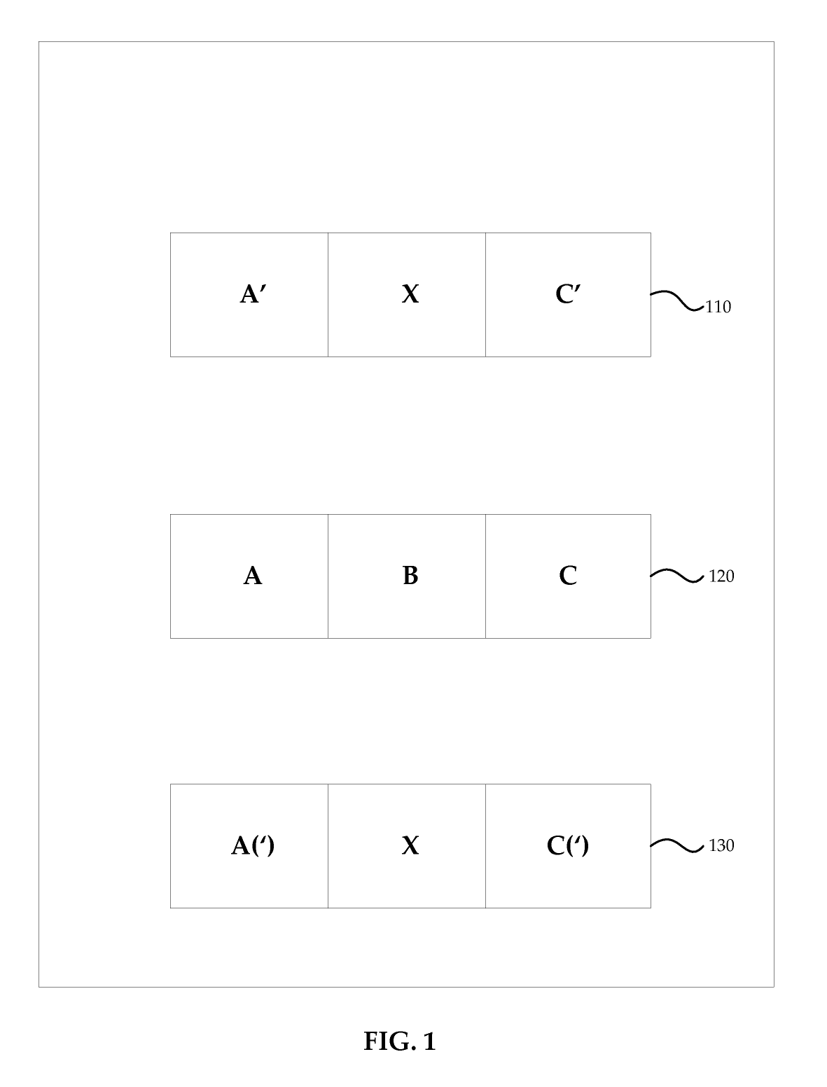

FIG. 1 is a diagram showing how exemplary deoxyribonucleic acid (DNA) sequences may be utilized for introducing DNA into the nucleus of an algal cell, according to one exemplary embodiment. Shown in FIG. 1 is a transformation construct 110, algal nuclear DNA 120, and transformed algal nuclear DNA 130.

The transformation construct 110 comprises a first sequence of DNA A′ that is similar in length and sequence to a corresponding first sequence of algal nuclear DNA A, as found in the algal nuclear DNA 120. The transformation construct 110 comprises a second sequence of DNA C′ that is similar in length and sequence to a corresponding second sequence of the nuclear DNA C as found in the algal nuclear DNA 120. The transformation construct 110 further comprises a sequence of DNA of interest X that is inserted between the first A′ and second C′ sequences of DNA of the transformation construct 110.

In one exemplary method for introducing DNA into the nucleus of an algal cell, a transformation construct such as exemplary transformation construct 110 is prepared. The transformation construct 110 may then be used to transform a target sequence of DNA B inserted between the first A and second C sequences of the nuclear DNA 120, resulting in replacement of the target sequence of DNA B with the sequence of DNA of interest X.

According to various exemplary embodiments, the first A′ and/or the second C′ sequences of DNA similar to the corresponding respective first A and/or the second C sequences of the nuclear DNA 120 may be of any length in base pairs (bps), ranging from approximately 0 bps to approximately 10,000 (bps), or longer. Additionally, the first sequence of DNA A′ may or may not have a length in base pairs equal to a length in base pairs of the second sequence of DNA C′.

In various exemplary embodiments, the target sequence of DNA B inserted between the first A and second C sequences of the nuclear DNA 120 may be of any length in base pairs, ranging from approximately 0 bps to approximately 10,000 (bps), or longer.

According to some exemplary embodiments, the sequence of DNA of interest X may separate the first A′ and second C′ sequences of the transformation construct 110 by as few as approximately 0 (bps) to as many as approximately 10,000 (bps). The sequence of DNA of interest X may comprise various sequences, such as a regulatory or promoter sequence (uni-directional or bi-directional), an antibiotic resistance marker, or may comprise a promoter sequence and an antibiotic resistance marker. In other exemplary embodiments, the sequence of DNA of interest X may comprise a gene for nutrient assimilation or biosynthesis of a metabolite. For instance, the sequence of DNA of interest X may comprise a gene coding for nitrate reductase or nitrite reductase.

In various exemplary embodiments, the sequence of DNA of interest X may or may not encode at least a portion of a polypeptide. In some cases, the sequence of DNA of interest X may only be transcribed, however not translated as a polypeptide. In other embodiments, the sequence of DNA of interest X may encode a peptide that is added to a peptide encoded by either the first A or the second C sequence of the nuclear DNA 120. The sequence of DNA of interest X may also encode a non-coding regulatory DNA sequence. In various exemplary embodiments, the sequence of DNA of interest X may not be similar in length to the target sequence of DNA B on the nuclear DNA 120. For instance, the sequence of DNA of interest X may be approximately 0 (bps) in length, resulting in deletion or near deletion of the target sequence of DNA B, as may be observed in the transformed algal nuclear DNA 130.

According to some exemplary embodiments, the transformation construct 110 may be used to transform a target sequence of DNA B inserted between the first A and second C sequences of the nuclear DNA 120, resulting in replacement of the target sequence of DNA B with the sequence of DNA of interest X. The nuclear DNA 120 may be at least a portion of a genome from the algal genus Nannochloropsis. Further, the genome of the algal genus Nannochloropsis may be a haploid genome. The transformation methodologies described herein may be used to change a phenotypic characteristic of an algal cell to impart new characteristics to the algal cell. For instance, the replacement of the target sequence of DNA B with the sequence of DNA of interest X may be at least a partial replacement, resulting in a partial decrease in gene function of the target sequence of DNA. In other embodiments, the sequence of DNA of interest X may comprise DNA to compromise or destroy wild-type functioning of the target gene B gene, which is otherwise needed for nutrient assimilation or biosynthesis of a metabolite. Conversely, the sequence of DNA of interest X may be used to transform the compromised or destroyed wild-type functioning of the gene for nutrient assimilation or biosynthesis back to wild-type functioning. For instance, the sequence of DNA of interest X may transform an auxotrophic algal cell, resulting in assimilation or biosynthesis of a metabolite. Such transformants may be selected via cultivation in a liquid or solid media that does not include the metabolite required for growth of the transformed auxotrophic algal cell.

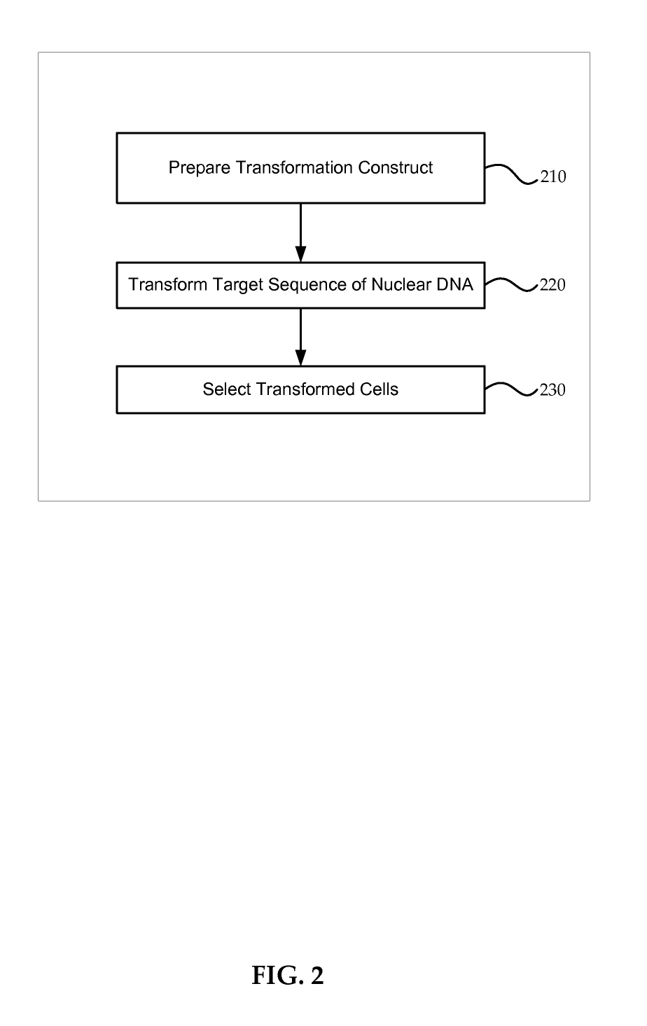

FIG. 2 is a flow chart showing an exemplary method for homologous recombination in an algal nuclear genome.

At step 210, a transformation construct is prepared. In one exemplary embodiment, the transformation construct 110 (FIG. 1) comprises a first sequence of DNA A′ that is similar to a corresponding first sequence of algal nuclear DNA A as found in the algal nuclear DNA 120 (FIG. 1). The transformation construct 110 may also comprise a second sequence of DNA C′ that is similar to a corresponding second sequence of the nuclear DNA C as found in the algal nuclear DNA 120. The transformation construct 110 may have a sequence of DNA of interest X inserted between the first A′ and second C′ sequences of DNA of the transformation construct 110.

At step 220, a target sequence of nuclear DNA is transformed. According to various exemplary embodiments, the transformation construct 110 is used to transform a target sequence of DNA B inserted between the first A and second C sequences of the nuclear DNA 120, resulting in replacement of the target sequence of DNA B with the sequence of DNA of interest X.

At step 230, transformed cells are selected. For instance, the sequence of DNA of interest X may transform an auxotrophic algal cell, resulting in assimilation or biosynthesis of a metabolite. Such transformants may be selected via cultivation in a liquid or solid media that does not include the metabolite required for growth of the transformed auxotrophic algal cell.

### Example 1

In order to test the possibility of homologous recombination in Nannochloropsis, the inventors created a transformation construct which utilized a selectable marker (a bleomycin gene) flanked by a left and a right nitrate reductase DNA sequence.

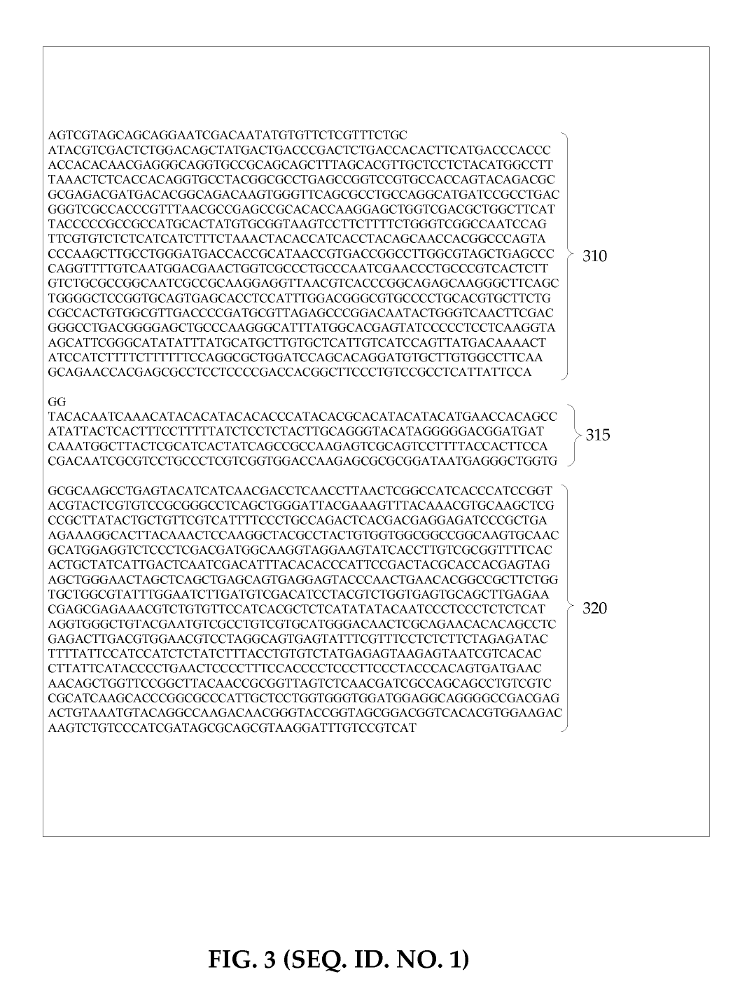

FIG. 3 shows an exemplary DNA sequence (SEQ. ID. NO. 1), which includes at least a portion of a nitrate reductase gene.

Referring to FIG. 3, a left nitrate reductase DNA sequence is designated 310, and a right nitrate reductase DNA sequence is designated 320. As will be described herein, a DNA sequence 315 between flanks 310 and 320 will be displaced from the endogenous nitrate reductase gene with DNA sequences from the transformation construct.

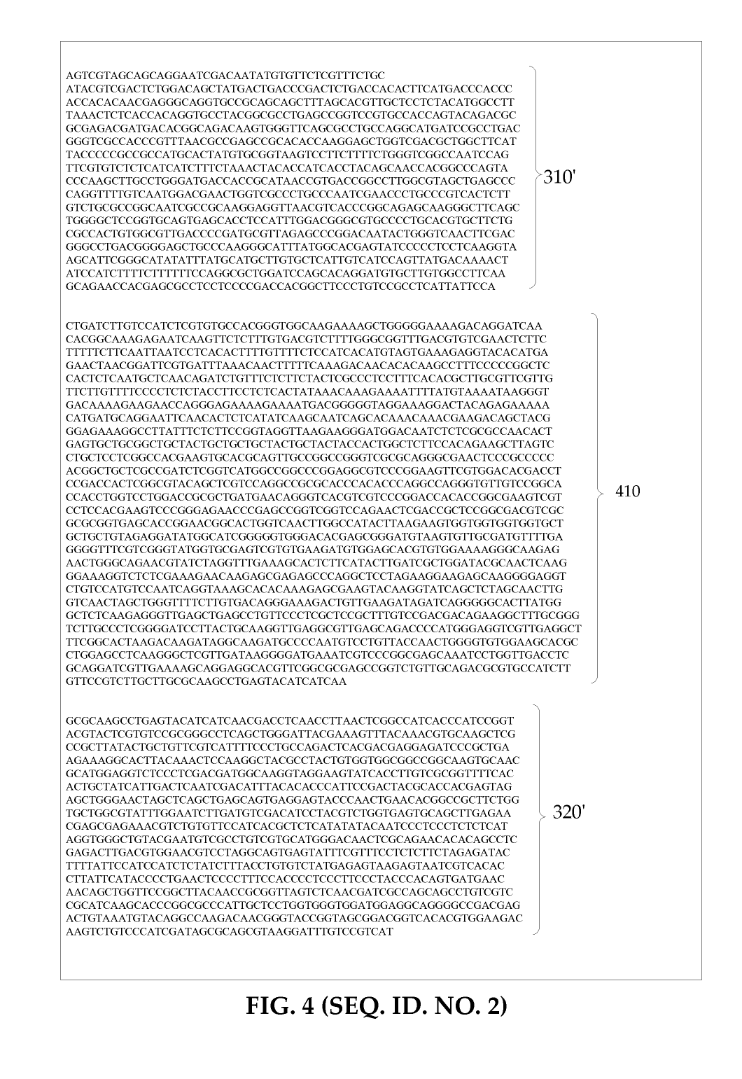

FIG. 4 shows an exemplary transformation construct (SEQ. ID. NO. 2), which incorporates the nitrate reductase DNA sequences used to create the flanks of the transformation construct. FIG. 4 shows the left nitrate reductase DNA sequence 310′, a selection cassette NT7 410, and the right nitrate reductase DNA sequence 320′. The selection cassette NT7 410 comprises a Violaxanthin-chlorophyll a binding protein (“Vcp”) 3′ UTR, a bleomycin resistance sequence, and a Vcp promoter sequence. The Vcp promoter and the Vcp 3′UTR DNA sequences were obtained from 2 different Vcp gene clusters, as described in U.S. Non-Provisional patent application Ser. No. 12/480,635 filed on Jun. 8, 2009, titled “VCP-Based Vectors for Algal Cell Transformation. The NT7-cassette comprising the Vcp promoter, bleomycin resistance sequence, and Vcp 3′ UTR were inserted in an anti-parallel fashion relative to the left nitrate reductase flank 310′ and the right nitrate reductase flank 320′.

Design.

Primers Used.

Homologous recombination of Vcp ble UTR into NR, reverse direction and deletion of part of one exon

P311 NR LEFT for
AGTCGTAGCAGCAGGAATCGACAA.
P312 NR LEFT rev
GGCACACGAGATGGACAAGATCAGTGGAATAATGAGGCGGACAGGGAA.
P313 NR RIGHT for
GTGCCATCTTGTTCCGTCTTGCTTGCGCAAGCCTGAGTACATCATCAA.
P314 NR RIGHT rev
ATGACGGACAAATCCTTACGCTGC.
P215 NT7 comp for
AAGCAAGACGGAACAAGATGGCAC.
P119 PL38 3UTR BACK
CTGATCTTGTCCATCTCGTGTGCC.

PCRs were performed with Takara Taq to generate NR flanks and insertion cassette:

P311×P312 on gDNA for Left flank LF (1 kB).

P313×P314 on gDNA for Right flank RF (1.04 kB).

(NOTE: both flanks contain fusion areas to NT7 derived from primer 312 and 313).

P215×P119 on NT7 for Insertion construct IC (1.817 kB).

All PCR products were then gel purified.

The LF, IC and RF fragments were linked with the following PCRs:

ALL 100 μl PCR RXNs

170 ng of LF+170 ng IC were used in fusion PCR with P311×P215 (2.817 kB)LF-IC.

170 ng of RF+170 ng IC were used in fusion PCR with P119×P314 (2.821 kB)RF-IC.

Fragments were gel purified and used for last PCR.

170 ng LF-IC+170 ng RF-IC with P311×P314.

3.8 kB DNA Fragment recovered from gel and directly used for transformation.

Transformation.

200 ng DNA fragment (see above) were used in the previously described transformation protocol.

Differences: cells were grown in NH4CL-containing F2 media (2 mM NH4Cl instead of nitrate). Recovery after transformation before plating was also done in 2 mM NH4Cl medium.

Cells were plated on F2 (zeocine-containing) plates with 2 mM NH4CL (instead of 2 mM NO3-). All media in 50% salinity compared to seawater.

Selection.

200 colonies were picked, resuspended in 100 μl nitrogen-deficient F2 media and spotted on Square plates (F2 media) with different nitrogen sources:

No Nitrogen

2 mM No2-

2 mM NO3-

2 mM NH4Cl

The overwhelming majority of these colonies could not grow on nitrate (turned yellowish indicating nitrogen starvation; nitrate reductase knock-out mutants cannot grow on nitrate as the sole nitrogen source), but all clones grew equally well on nitrite and ammonium-chloride plates. Further, appearance of those clones suppressed in growth on nitrate was indistinguishable from cells (transformed or untransformed) grown on nitrogen-deficient (no nitrogen) plates indicating that the growth retardation of mutants on nitrate is due to an inability to use nitrate as a nitrogen source. Growth retardation on agar plates containing nitrate as the sole nitrogen source was never observed with wild types nor with mutants obtained from nitrate reductase unrelated transformation, indicating that the clones were inactivated within the nitrate reductase gene.

Results.

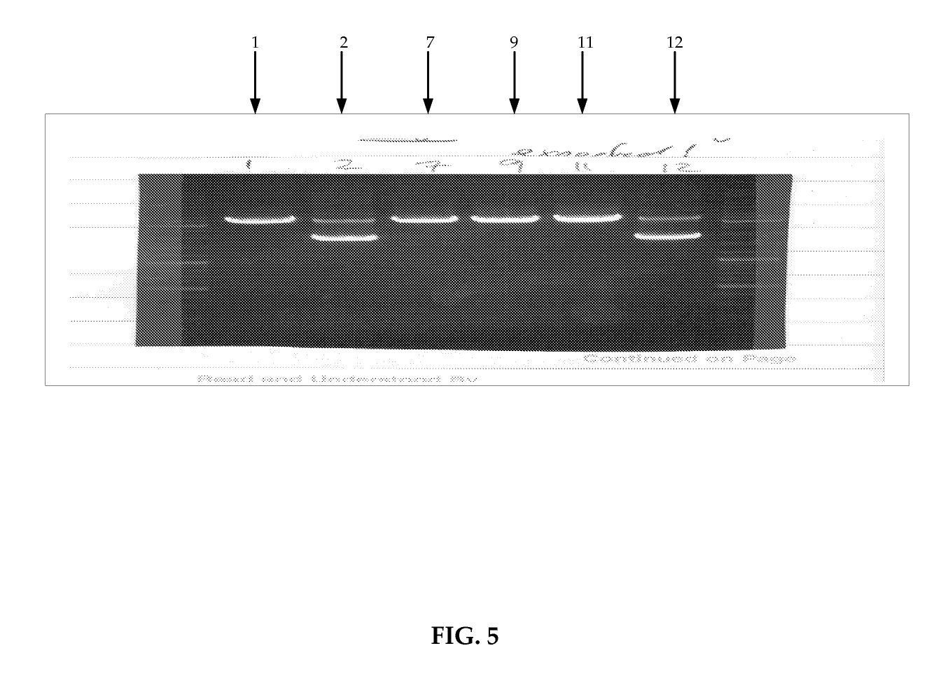

FIG. 5 is a gel showing a PCR analysis of several transformants obtained with the transformation construct illustrated in FIG. 4.

192 clones were analyzed. 176 of these were apparently nitrate reductase deficient via visual screening. Colonies were also analyzed via PCR. The gel in FIG. 5 shows the molecular genetic analysis of several transformants (designated 1, 2, 7, 9, 11 and 12). Clones 2 and 12 have been identified to grow on nitrate as a sole nitrogen source, while clones 1, 7, 9 and 11 could not, indicating a disruption of the nitrate reductase gene.

The primer used for genetic analysis via PCR would yield a smaller DNA fragment for the wild-type gene and a larger DNA fragment for a mutant gene which contains the large selection marker insertion.

The lanes labeled 1, 7, 9 and 11 show only one band that corresponds to the nitrate reductase locus with the expected insert. Lanes labeled 2 and 12 show two bands—the smaller band is the endogenous nitrate reductase gene, and the larger band is the transformation construct fragment, which is inserted somewhere else in the genome but not within the nitrate reductase locus.

Sequencing.

Sequencing was employed to verify if there were errors introduced after recombination. 6 clones were analyzed via PCR, and the flanking regions including the flank ends (5′ end of left flank and 3′ end of right flank) were sequenced. No error could be found. The entire locus has also been amplified out of transformants (nitrate reductase interrupted by ble gene cassette) and successfully used for repeated transformations of wild-type.

The inventors were also successful using a wild-type nitrate reductase fragment as a selection marker to rescue a knock out mutant by homologous recombination: the wild-type fragment patched over the insertion site of the ble gene within the nitrate reductase gene and replaced it.

Only those clones, in which the nitrate reductase gene was rescued by homologous recombination, could grow on nitrate as the sole nitrogen source.

### Example 2

Our model organism, Nannochloropsis sp. (strain W2J3B), grows rapidly on solid or liquid media containing nitrate, nitrite, or ammonia as the sole nitrogen source, and it has a relatively small genome size of approximately 30 Mb. We identified strong promoters and constructed transformation vectors based on selection markers conferring resistance to zeocin, hygromycin B, or blastocidin S. We developed and optimized an efficient transformation method based on electroporation, allowing the generation of thousands of transformants in a single experiment (approximately 2500 transformants per microgram of DNA). We also performed experiments in which we transformed the zeocin-resistance vector together with the hygromycin B- and/or blastocidin S-resistance markers and plated the cells on zeocin only. Replating of zeocin-resistant colonies on hygromycin B and/or blastocidin S selective media revealed a high cotransformation frequency of 72% or 22% for one or both unselected markers, respectively.

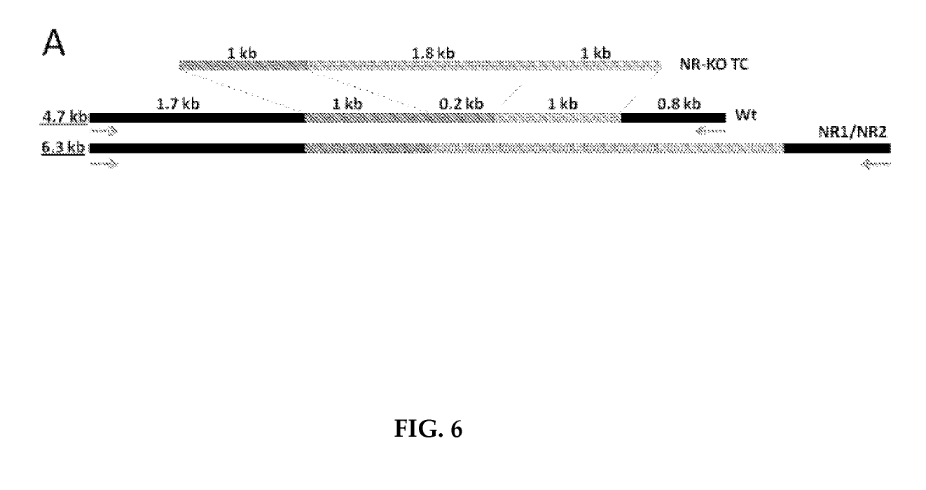

FIG. 6 shows the knock-out (“KO”) of a nitrate reductase (“NR”) gene by homologous recombination in Nannochloropsis sp. Structures of NR-KO transformation constructs (“TC”), wild-type (Wt) genes, and homologous recombination (“HR”) products are also shown.

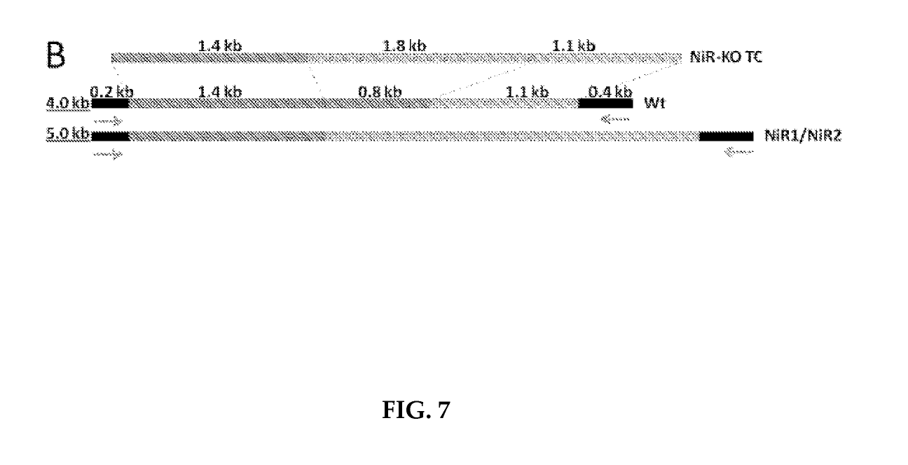

FIG. 7 shows the knock-out (“KO”) of a nitrite reductase (“NiR”) gene by homologous recombination in Nannochloropsis sp. Structures of NiR-KO transformation constructs (“TC”), wild-type (Wt) genes, and homologous recombination (“HR”) products are also shown.

To test the frequency of homologous recombination in Nannochloropsis sp., we performed transformation with knock-out (KO) constructs based on the zeocin-resistance cassette with approximately 1 kb flanking sequences targeting the nitrate reductase (“NR”) (FIG. 6) and nitrite reductase (“NiR”) (FIG. 7) genes, which are involved in nitrogen assimilation. In both cases we obtained zeocin-resistant transformants on medium containing ammonia as the sole nitrogen source.

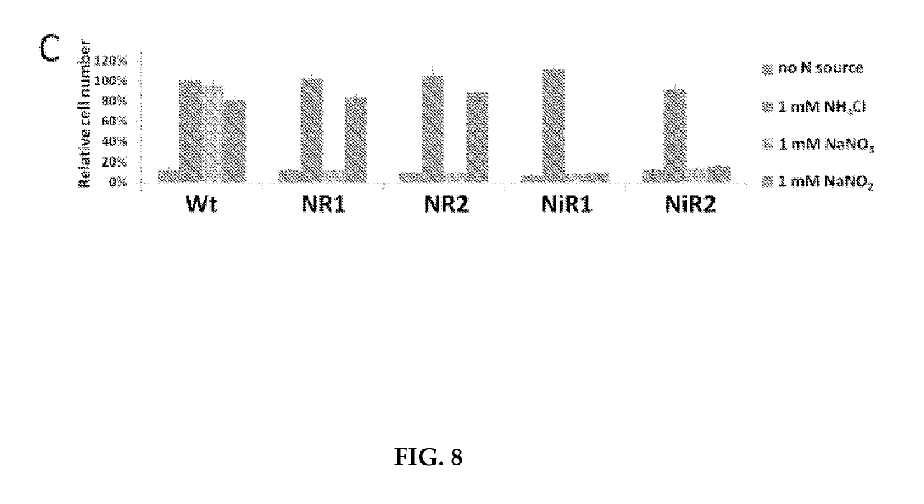

Replating of these colonies on media containing nitrate or ammonia as sole nitrogen source revealed that up to 95% of the transformants bleached on nitrate, whereas all transformants grew on ammonia. Each two of these clones bleaching on nitrate (two putative NR-KO mutants NR1 and NR2 and two putative NiR-KO mutants NiR1 and NiR2) have been analyzed further. FIG. 8 shows growth of Wt, 2 NR-KO mutants (NR1 and NR2), and two NiR-KO mutants (NiR1 and NiR2) with different nitrogen sources, relative to Wt in 1 mM NH4Cl.

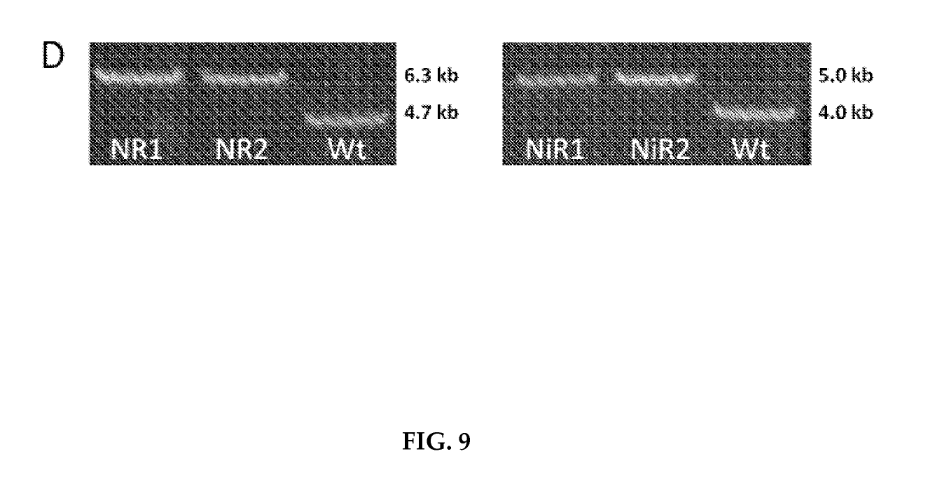

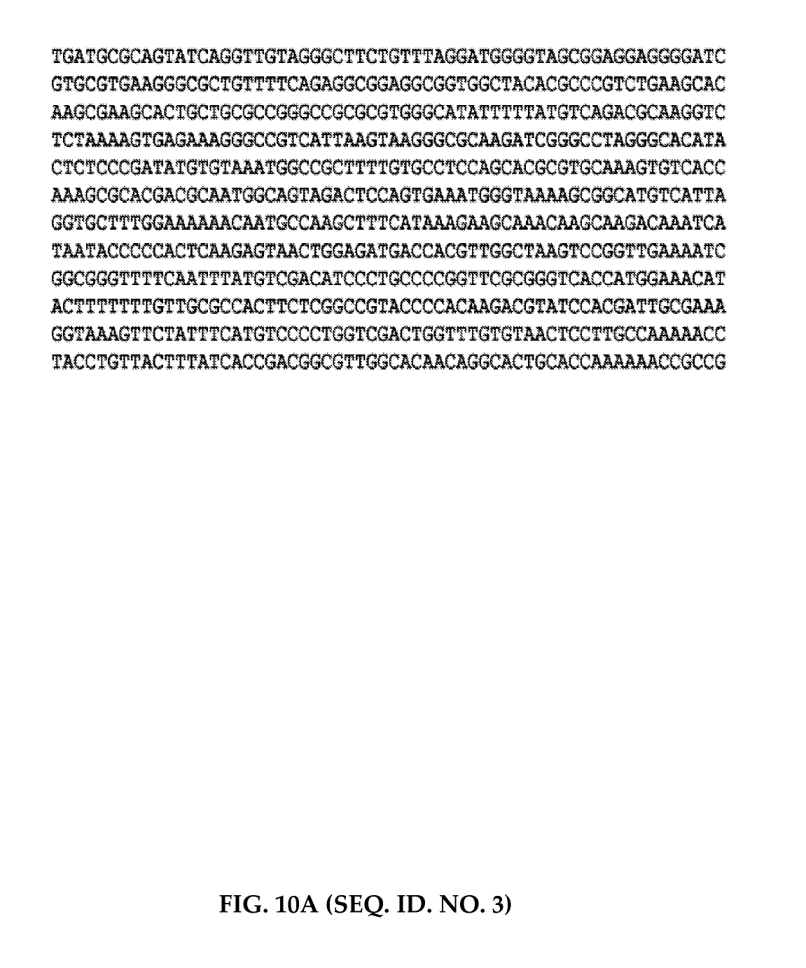

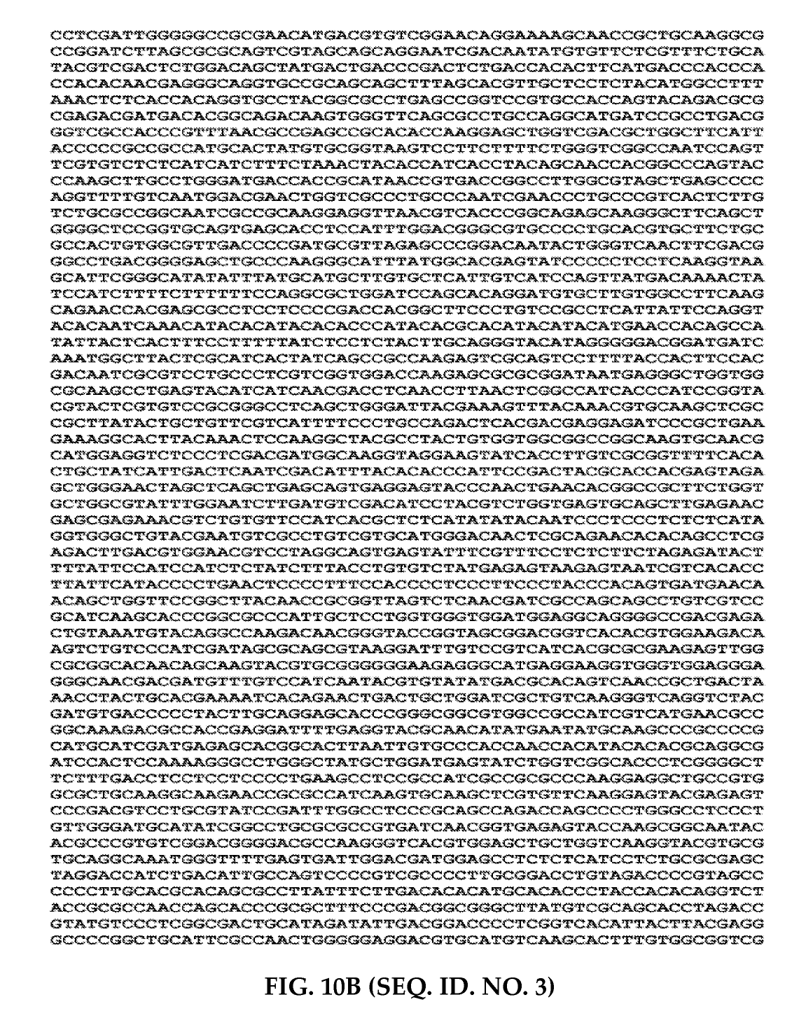

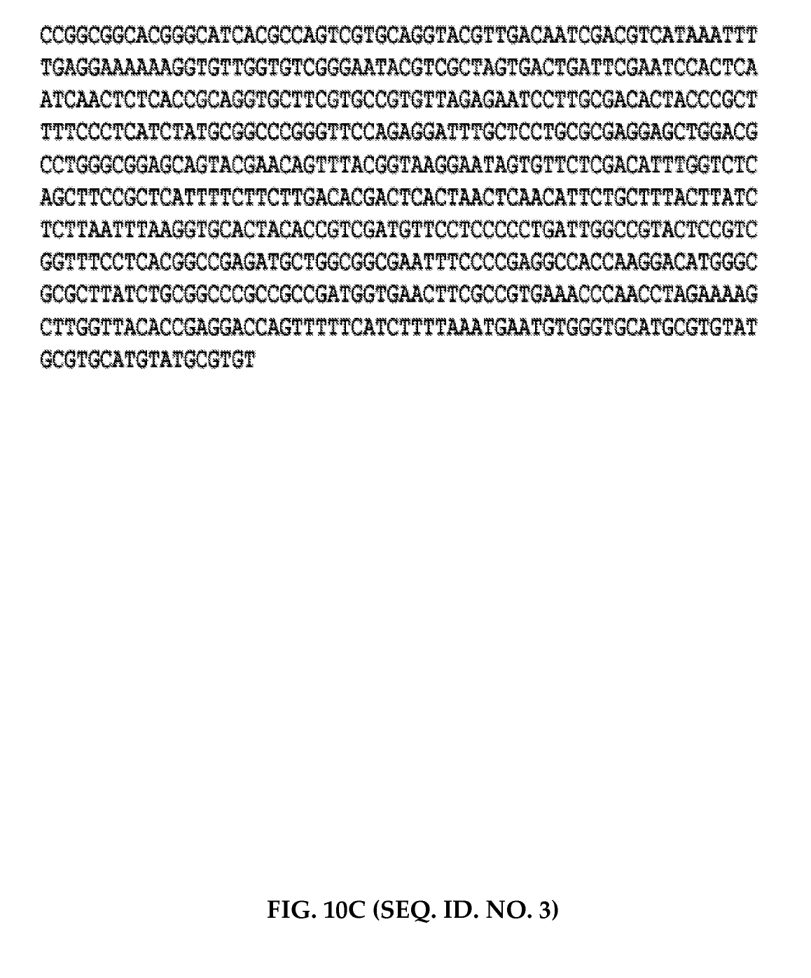

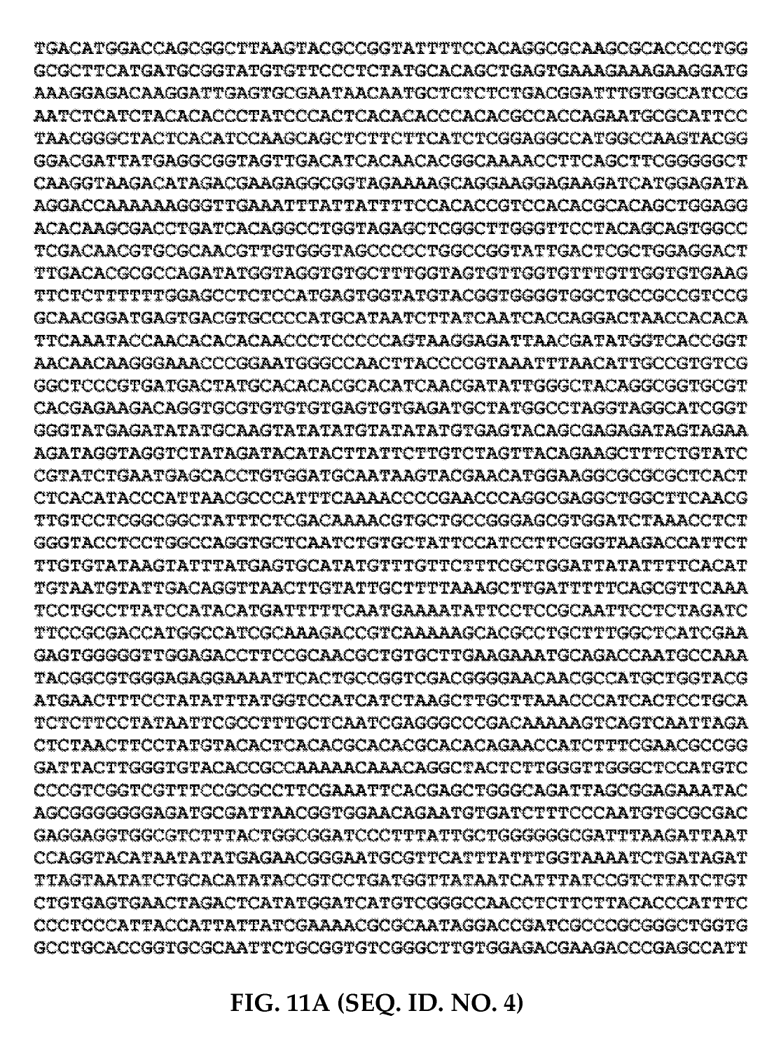

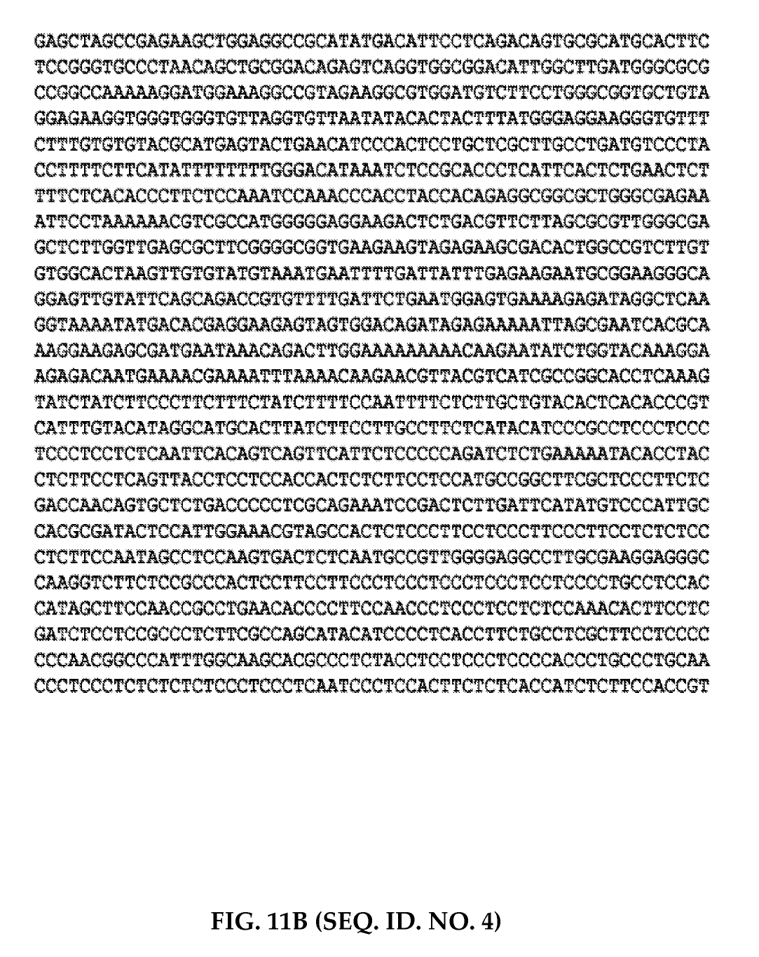

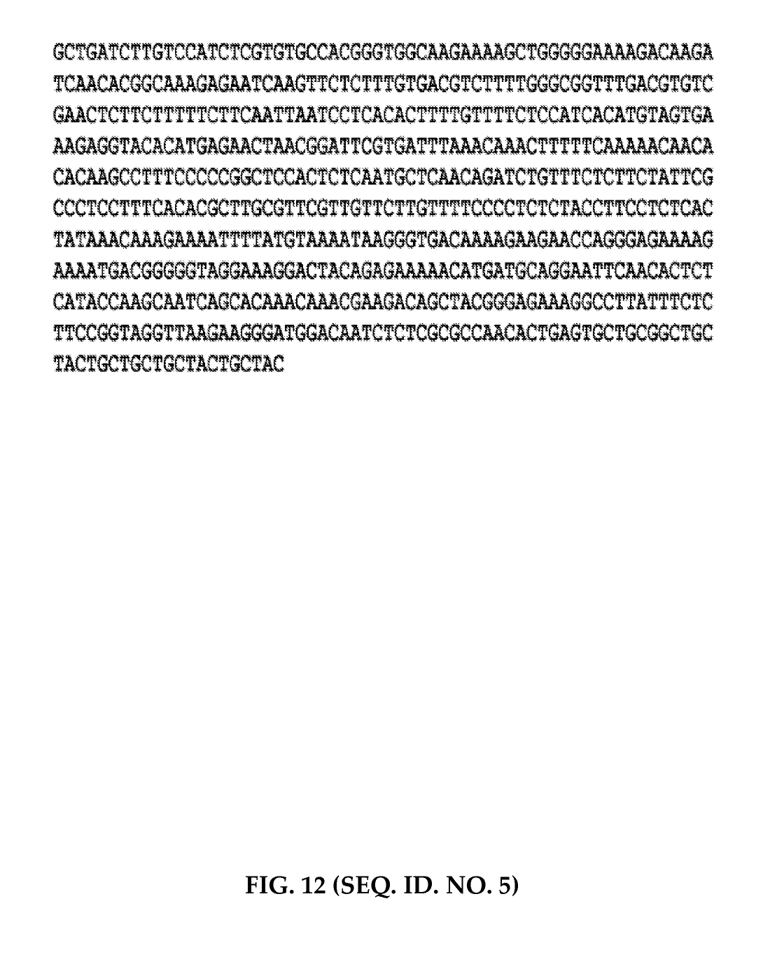

Liquid growth analysis of transformants that bleached on nitrate revealed that NR-KO transformants could not utilize nitrate and NiR-KO transformants could not utilize nitrate or nitrite as a nitrogen source. [001] FIG. 9 shows PCR analysis of NR-KO and NiR-KO transformants. PCR analysis of the genomic DNA of transformants revealed that the KO construct had successfully inserted into the genome and replaced part of the target gene with the selectable marker. The presence of a single PCR product and the absence of the wild-type allele strongly suggest that Nannochloropsis sp. W2J3B is haploid. FIG. 8 shows growth of Wt, 2 NR-KO mutants (NR1 and NR2), and two NiR-KO mutants (NiR1 and NiR2) with different nitrogen sources, relative to Wt in 1 mM NH4Cl.

FIG. 9 shows PCR analysis of NR-KO and NiR-KO transformants. PCR analysis of the genomic DNA of transformants revealed that the KO construct had successfully inserted into the genome and replaced part of the target gene with the selectable marker. The presence of a single PCR product and the absence of the wild-type allele strongly suggest that Nannochloropsis sp. W2J3B is haploid.

Material and Methods

Growth conditions. Nannochloropsis sp. W2J3B was grown in F2N medium: 50% artificial seawater (16.6 g/L Instant Ocean) supplemented with 0.72 mM NaH₂PO₄*H₂O, 24 μM FeCl₃*6H₂O, 125 μM Na₂EDTA, 0.2 μM CuSO₄*5H₂O, 0.13 μM Na₂MoO₄*2H₂O, 0.38 μM ZnSO₄*7 H₂O, 0.24 μM CoCl₂*6H₂O, 4.5 μM MnCl₂*4H₂O, 20.5 nM Biotin, 3.7 nM Vitamin B12, 14.8 nM Thiamin HCl, 10 mM Tris-HCl, pH 7.6. 5 mM NH₄Cl was included as a nitrogen source. All chemicals were obtained from Sigma as reagent grade. Agar plates were prepared with 0.8% Bacto agar (Difco) in F/2 medium {Guillard and Ryther, 1962} with 50% artificial seawater, except that 2 mM NH₄Cl was used as a nitrogen source. Zeocin, Blastocidin S, or Hygromycin B, if needed, was added to a final concentration of 2 μg/mL, 50 μg/mL, or 300 μg/mL, respectively. Liquid cultures were generally maintained in F2N medium at a photon flux density of 85 μmol photons m⁻² s⁻¹ and bubbled with CO₂-enriched air (3% CO₂) at 28° C. Agar plates were maintained at the same light intensity at 26° C.

Nucleic acids used for transformation. For polymerase chain reaction (PCR) we used the Takara LA Taq polymerase. Two overlapping PCR products containing the Sh ble gene were amplified from pTEF1/Zeo (Invitrogen) via primer pair 5′-ATGGCCAAGTTGACCAGTGCCGT-3′ and 5′-TTAGTCCTGCTCCTCGGCCACGAA-3′ and primer pair 5′-ATGGCCAAGTTGACCAGTGCCGT-3′ and 5′-ACAGAAGCTTAGTCCTGCTCCTCGGCCACGAA-3′ (phosphorylated). The resulting products with different lengths were gel purified (QiaEx II; Qiagen)), mixed in equimolar amounts, denatured, and allowed to anneal at RT. Similarly, two overlapping products containing the 3′ UTR of the VCP1 gene were amplified from genomic DNA of Nannochloropsis sp. W2J3B with primer pair 5′-CTGATCTTGTCCATCTCGTGTGCC-3′ and 5′-GCTTCTGTGGAAGAGCCAGTGGTAG-3′ and primer pair 5′-CTGATCTTGTCCATCTCGTGTGCC-3′ and 5′-GGAAGAGCCAGTGGTAGTAGCAGT-3′. These products were also gel purified, mixed in equimolar amounts, denatured, and allowed to anneal at RT. The products of the two annealing reactions were ligated for 1 h with T4 Ligase (Fermentas) to generate the product ble^{uTR}, which was then gel purified and amplified with primers 5′-ATGGCCAAGTTGACCAGTGCCGTTCC-3′ (phosphorylated) and 5′-CTGATCTTGTCCATCTCGTGTGCC-3′ and gel purified. Primers 5′-ACTTAAGAAGTGGTGGTGGTGGTGC-3′ and 5′-ACTTGAGAGAGTGGTGGAGTTGACT-3′ were used to amplify the bidirectional VCP2 promoter (VCP2^{Prom}). The VCP2^{Prom} and ble^{UTR} products were blunt ligated, gel purified, cloned into the pJet1 vector (Fermentas), and transformed into E. coli DH5a cells. After re-isolation of plasmids and sequencing we obtained vectors pJet-C1 and pJet-C2, driving expression of the Sh ble gene from one side or the other of the bidirectional VCP2 promoter. The selection marker cassettes C2 or NT7 were amplified from pJet-C2 with primer pair 5′-ACTTAAGAAGTGGTGGTGGTGGTGC-3′ and 5′-CTGATCTTGTCCATCTCGTGTGCC-3′ or 5′-AAGCAAGACGGAACAAGATGGCAC-3′ and 5′-CTGATCTTGTCCATCTCGTGTGCC-3′, respectively. The difference between NT7 and C2 is that C2 contains the entire bidirectional promoter, whereas NT7 contains only the part driving expression of the sh ble gene.

For the nitrate reductase (NR) KO construct, we amplified two ˜1 kb parts of the NR gene separated by 242 bp within the genome as recombination flanks with the primers 5′-AGTCGTAGCAGCAGGAATCGACAA-3′ and 5′-GGCACACGAGATGGACAAGATCAGTGGAATAATGAGGCGGACAGGGAA-3′ (NR left flank), and 5′-GTGCCATCTTGTTCCGTCTTGCTTGCGCAAGCCTGAGTACATCATCAA-3′ and 5′-ATGACGGACAAATCCTTACGCTGC-3′ (NR right flank). Flanks were constructed for the nitrite reductase (NiR) gene by amplifying left and right flanks (separated by 793 bp within the genome) with the primers 5′-TGACATGGACCAGCGGCTTAAGTA-3′ and 5′-GTGCCATCTTGTTCCGTCTTGCTTGCCGTATTTGGCATTGGTCTGCAT-3′ (NiR left flank), and 5′-GGCACACGAGATGGACAAGATCAGAGGCCGCATATGACATTCCTCAGA-3′ and 5′-ACGGTGGAAGAGATGGTGAGAGAA-3′ (NiR right flank). Flanks derived from the NR or NiR gene were fused to the NT7 transformation cassette by a fusion PCR utilizing the primers 5′-AGTCGTAGCAGCAGGAATCGACAA-3′ and 5′-ATGACGGACAAATCCTTACGCTGC-3′ or 5′-TAACGGGCTACTCACATCCAAGCA-3′ and 5′-AGTATCGCGTGGCAATGGGACATA-3′, respectively. The resulting PCR products (NR-KO and NiR-KO, respectively) were gel purified prior to transformation.

Nuclear transformation of Nannochloropsis sp. W2J3B. Cells were grown in F2N medium to mid-log phase and washed four times in 384 mM D-sorbitol. Cell concentration was adjusted to 10¹⁰ cells/ml in 384 mM D-sorbitol, and 100 μL cells and 1 μg DNA were used for each electroporation within an hour. Electroporation was performed with a Biorad Gene Pulser I Electroporator in 2 mm cuvettes. The electroporator was adjusted to exponential decay, 2200 V field strength, 50 μF capacitance, and 500 Ohm shunt resistance. After electroporation, cells were immediately transferred into 15 mL conical falcon tubes containing 10 mL F2N medium and were incubated in low light overnight. 5×10⁸ cells were plated the next day on F/2 square agar plates (500 cm² area) containing 2 μg/mL zeocin. Colonies appeared after 2 weeks and could be further processed after 3 weeks.

Screening and analysis of knock out (KO) mutants. Initial screen: Clones obtained by transformation with either NiR-KO or NR-KO were spotted on agar plates containing 1 mM KNO₃ or 1 mM NH₄Cl as a sole nitrogen source. Many clones started to bleach on plates containing nitrate indicating starvation for a nutrient, whereas no signs of starvation were visible on plates containing NH₄Cl. Randomly picked clones showing bleaching were subjected to further analysis.

PCR screen: Genomic DNA from randomly chosen clones was isolated, and PCR with primers 5′-ACACGCATACATGCACGCATACAC-3′ and 5′-TGATGCGCAGTATCAGGTTGTAGG-3′ on NR-KO mutants and with primers 5′-TGACATGGACCAGCGGCTTAAGTA-3′ and 5′-ACGGTGGAAGAGATGGTGAGAGAA-3′ on NiR-KO mutants was used to amplify the genomic DNA around the NR or NiR gene, respectively. PCR on genomic DNA isolated from the wild type was used as a control.

Growth test: Wild type (Wt) and two clones each of NR and NiR KO mutants (NR1, NR2, NiR1, and NiR2) were grown to mid-log phase in F2N medium containing 1 mM NH₄Cl. Cells were washed three times with 50% artificial seawater by centrifugation (5 min, 3000 g) and subsequent resuspension of the cells. Beakers with a clear lid containing 100 mL of F2N medium with no nitrogen source, 1 mM KNO₃, 1 mM NaNO₂ or 1 mM NH₄Cl were inoculated in triplicate with washed cells to a concentration of 4×10⁵ cells/mL and allowed to grow under 3% CO₂ atmosphere at 200 μmol photons m⁻² s⁻¹ for 4 days under constant shaking (80 rpm). At this time, Wt cultures supplemented with 1 mM NH₄Cl reached stationary phase after exhausting the nitrogen source. Cells were counted with an Accuri C6 flow cytometer equipped with an Accuri C6 sampler in duplicates. Growth was estimated as % cells compared to Wt cultures grown in F2N medium containing 1 mM NH₄Cl.

While various embodiments have been described above, it should be understood that they have been presented by way of example only, and not limitation. Thus, the breadth and scope of a preferred embodiment should not be limited by any of the above-described exemplary embodiments.

What is claimed is:

1. A transformation method for introducing deoxyribonucleic acid (DNA) into the nucleus of an algal cell, the method comprising:
preparing a transformation construct, the transformation construct having a first sequence of DNA similar to a corresponding first sequence of nuclear DNA, the transformation construct having a second sequence of DNA similar to a corresponding second sequence of the nuclear DNA, the transformation construct having a sequence of DNA of interest inserted between the first and second sequences of DNA of the transformation construct, and
transforming a target sequence of DNA inserted between the first and second corresponding sequences of the nuclear DNA, resulting in replacement of the target sequence of DNA with the sequence of DNA of interest.

2. The method of claim 1, wherein the replacement of the target sequence of DNA with the sequence of DNA of interest is at least a partial replacement resulting in a partial decrease in gene function of the target sequence of DNA.

3. A transformation construct, the transformation construct having a first sequence of DNA similar to a corresponding first sequence of nuclear DNA of an algal cell, the transformation construct having a second sequence of DNA similar to a corresponding second sequence of nuclear DNA of the algal cell, and the transformation construct having a sequence of DNA of interest inserted between the first and second sequences of the transformation construct.

4. The method of claim 1, wherein each of the first and second sequences of DNA similar to the corresponding respective first and second sequences of the nuclear DNA comprises approximately 1000 base pairs (bps).

5. The method of claim 1, wherein each of the first and second sequences of DNA similar to the corresponding respective first and second sequences of the nuclear DNA comprises approximately less than 1000 bps.

6. The method of claim 1, wherein each of the first and second sequences of DNA similar to the corresponding respective first and second sequences of the nuclear DNA comprises approximately greater than 1000 bps.

7. The method of claim 1, wherein each of the first and second sequences of DNA similar to the corresponding respective first and second sequences of the nuclear DNA comprises approximately greater than 10,000 bps.

8. The method of claim 1, wherein the sequence of DNA of interest further comprises DNA to compromise or destroy wild-type functioning of a gene for nutrient assimilation or biosynthesis of a metabolite.

9. The method of claim 1, wherein the sequence of DNA of interest transforms an auxotrophic algal cell, resulting in assimilation or biosynthesis of a metabolite.

10. The method of claim 9, the method further comprising selecting the transformed auxotrophic algal cell via cultivation in media that does not include the metabolite required for growth of the transformed auxotrophic algal cell.

11. The method of claim 8, wherein the gene codes for nitrate reductase or nitrite reductase.

12. The method of claim 8, the method further comprising: transforming the compromised or destroyed wild-type functioning of the gene for nutrient assimilation or biosynthesis back to wild-type functioning.

13. The method of claim 8, wherein the sequence of DNA of interest separates the first and second sequences of DNA similar to the corresponding respective first and second sequence of the nuclear DNA by approximately 200 bps.

14. The method of claim 1, wherein the sequence of DNA of interest separates the first and second sequences of DNA similar to the corresponding respective first and second sequence of the nuclear DNA by approximately 10.0 kb.

15. The method of claim 1, wherein at least a portion of the sequence of DNA of interest encodes a polypeptide.

16. The method of claim 1, wherein either the first or second sequence of DNA similar to the corresponding respective first or second sequence of the nuclear DNA comprises a length in base pairs ranging from approximately 1 base pair to approximately 10,000 base pairs.

17. The method of claim 1, wherein the sequence of DNA of interest comprises a length in base pairs ranging from approximately 1 base pair to approximately 10,000 base pairs.

18. The method of claim 10, wherein the media is either solid or liquid.


SEQUENCE LISTING (5 sequences)

SEQ ID NO: 1 - DNA, 2247 bp, Nannochloropsis
agtcgtagcagcaggaatcgacaatatgtgttctcgtttctgcatacgtcgactctggacagctatgactgacccgactctgaccacacttcatgacccacccaccacacaacgagggcaggtgccgcagcagctttagcacgttgctcctctacatggcctttaaactctcaccacaggtgcctacggcgcctgagccggtccgtgccaccagtacagacgcgcgagacgatgacacggcagacaagtgggttcagcgcctgccaggcatgatccgcctgacgggtcgccacccgtttaacgccgagccgcacaccaaggagctggtcgacgctggcttcattacccccgccgccatgcactatgtgcggtaagtccttcttttctgggtcggccaatccagttcgtgtctctcatcatctttctaaactacaccatcacctacagcaaccacggcccagtacccaagcttgcctgggatgaccaccgcataaccgtgaccggccttggcgtagctgagccccaggttttgtcaatggacgaactggtcgccctgcccaatcgaaccctgcccgtcactcttgtctgcgccggcaatcgccgcaaggaggttaacgtcacccggcagagcaagggcttcagctggggctccggtgcagtgagcacctccatttggacgggcgtgcccctgcacgtgcttctgcgccactgtggcgttgaccccgatgcgttagagcccggacaatactgggtcaacttcgacgggcctgacggggagctgcccaagggcatttatggcacgagtatccccctcctcaaggtaagcattcgggcatatatttatgcatgcttgtgctcattgtcatccagttatgacaaaactatccatcttttcttttttccaggcgctggatccagcacaggatgtgcttgtggccttcaagcagaaccacgagcgcctcctccccgaccacggcttccctgtccgcctcattattccaggtacacaatcaaacatacacatacacacccatacacgcacatacatacatgaaccacagccatattactcactttcctttttatctcctctacttgcagggtacatagggggacggatgatcaaatggcttactcgcatcactatcagccgccaagagtcgcagtccttttaccacttccacgacaatcgcgtcctgccctcgtcggtggaccaagagcgcgcggataatgagggctggtggcgcaagcctgagtacatcatcaacgacctcaaccttaactcggccatcacccatccggtacgtactcgtgtccgcgggcctcagctgggattacgaaagtttacaaacgtgcaagctcgccgcttatactgctgttcgtcattttccctgccagactcacgacgaggagatcccgctgaagaaaggcacttacaaactccaaggctacgcctactgtggtggcggccggcaagtgcaacgcatggaggtctccctcgacgatggcaaggtaggaagtatcaccttgtcgcggttttcacactgctatcattgactcaatcgacatttacacacccattccgactacgcaccacgagtagagctgggaactagctcagctgagcagtgaggagtacccaactgaacacggccgcttctggtgctggcgtatttggaatcttgatgtcgacatcctacgtctggtgagtgcagcttgagaacgagcgagaaacgtctgtgttccatcacgctctcatatatacaatccctccctctctcataggtgggctgtacgaatgtcgcctgtcgtgcatgggacaactcgcagaacacacagcctcgagacttgacgtggaacgtcctaggcagtgagtatttcgtttcctctcttctagagatacttttattccatccatctctatctttacctgtgtctatgagagtaagagtaatcgtcacaccttattcatacccctgaactcccctttccacccctcccttccctacccacagtgatgaacaacagctggttccggcttacaaccgcggttagtctcaacgatcgccagcagcctgtcgtccgcatcaagcacccggcgcccattgctcctggtgggtggatggaggcaggggccgacgagactgtaaatgtacaggccaagacaacgggtaccggtagcggacggtcacacgtggaagacaagtctgtcccatcgatagcgcagcgtaaggatttgtccgtcat

SEQ ID NO: 2 - DNA, 3822 bp, Artificial sequence
  Chemically synthesized; Parts from seq 1 interrupted by an artificial transformation construct
agtcgtagcagcaggaatcgacaatatgtgttctcgtttctgcatacgtcgactctggacagctatgactgacccgactctgaccacacttcatgacccacccaccacacaacgagggcaggtgccgcagcagctttagcacgttgctcctctacatggcctttaaactctcaccacaggtgcctacggcgcctgagccggtccgtgccaccagtacagacgcgcgagacgatgacacggcagacaagtgggttcagcgcctgccaggcatgatccgcctgacgggtcgccacccgtttaacgccgagccgcacaccaaggagctggtcgacgctggcttcattacccccgccgccatgcactatgtgcggtaagtccttcttttctgggtcggccaatccagttcgtgtctctcatcatctttctaaactacaccatcacctacagcaaccacggcccagtacccaagcttgcctgggatgaccaccgcataaccgtgaccggccttggcgtagctgagccccaggttttgtcaatggacgaactggtcgccctgcccaatcgaaccctgcccgtcactcttgtctgcgccggcaatcgccgcaaggaggttaacgtcacccggcagagcaagggcttcagctggggctccggtgcagtgagcacctccatttggacgggcgtgcccctgcacgtgcttctgcgccactgtggcgttgaccccgatgcgttagagcccggacaatactgggtcaacttcgacgggcctgacggggagctgcccaagggcatttatggcacgagtatccccctcctcaaggtaagcattcgggcatatatttatgcatgcttgtgctcattgtcatccagttatgacaaaactatccatcttttcttttttccaggcgctggatccagcacaggatgtgcttgtggccttcaagcagaaccacgagcgcctcctccccgaccacggcttccctgtccgcctcattattccactgatcttgtccatctcgtgtgccacgggtggcaagaaaagctgggggaaaagacaggatcaacacggcaaagagaatcaagttctctttgtgacgtcttttgggcggtttgacgtgtcgaactcttctttttcttcaattaatcctcacacttttgttttctccatcacatgtagtgaaagaggtacacatgagaactaacggattcgtgatttaaacaactttttcaaagacaacacacaagcctttcccccggctccactctcaatgctcaacagatctgtttctcttctactcgccctcctttcacacgcttgcgttcgttgttcttgttttcccctctctaccttcctctcactataaacaaagaaaattttatgtaaaataagggtgacaaaagaagaaccagggagaaaagaaaatgacgggggtaggaaaggactacagagaaaaacatgatgcaggaattcaacactctcatatcaagcaatcagcacaaacaaacgaagacagctacgggagaaaggccttatttctcttccggtaggttaagaagggatggacaatctctcgcgccaacactgagtgctgcggctgctactgctgctgctactgctactaccactggctcttccacagaagcttagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatacttaagaagtggtggtggtggtgctgctgctgtagaggatatggcatcgggggtgggacacgagcgggatgtaagtgttgcgatgttttgaggggtttcgtcgggtatggtgcgagtcgtgtgaagatgtggagcacgtgtggaaaagggcaagagaactgggcagaacgtatctaggtttgaaagcactcttcatacttgatcgctggatacgcaactcaagggaaaggtctctcgaaagaacaagagcgagagcccaggctcctagaaggaagagcaaggggaggtctgtccatgtccaatcaggtaaagcacacaaagagcgaagtacaaggtatcagctctagcaacttggtcaactagctgggttttcttgtgacagggaaagactgttgaagatagatcagggggcacttatgggctctcaagagggttgagctgagcctgttccctcgctccgctttgtccgacgacagaaggctttgcgggtcttgccctcggggatccttactgcaaggttgaggcgttgagcagaccccatgggaggtcgttgaggctttcggcactaagacaagataggcaagatgccccaatgtcctgttaccaactggggtgtggaagcacgcctggagcctcaagggctcgttgataaggggatgaaatcgtcccggcgagcaaatcctggttgacctcgcaggatcgttgaaaagcaggaggcacgttcggcgcgagccggtctgttgcagacgcgtgccatcttgttccgtcttgcttgcgcaagcctgagtacatcatcaagcgcaagcctgagtacatcatcaacgacctcaaccttaactcggccatcacccatccggtacgtactcgtgtccgcgggcctcagctgggattacgaaagtttacaaacgtgcaagctcgccgcttatactgctgttcgtcattttccctgccagactcacgacgaggagatcccgctgaagaaaggcacttacaaactccaaggctacgcctactgtggtggcggccggcaagtgcaacgcatggaggtctccctcgacgatggcaaggtaggaagtatcaccttgtcgcggttttcacactgctatcattgactcaatcgacatttacacacccattccgactacgcaccacgagtagagctgggaactagctcagctgagcagtgaggagtacccaactgaacacggccgcttctggtgctggcgtatttggaatcttgatgtcgacatcctacgtctggtgagtgcagcttgagaacgagcgagaaacgtctgtgttccatcacgctctcatatatacaatccctccctctctcataggtgggctgtacgaatgtcgcctgtcgtgcatgggacaactcgcagaacacacagcctcgagacttgacgtggaacgtcctaggcagtgagtatttcgtttcctctcttctagagatacttttattccatccatctctatctttacctgtgtctatgagagtaagagtaatcgtcacaccttattcatacccctgaactcccctttccacccctcccttccctacccacagtgatgaacaacagctggttccggcttacaaccgcggttagtctcaacgatcgccagcagcctgtcgtccgcatcaagcacccggcgcccattgctcctggtgggtggatggaggcaggggccgacgagactgtaaatgtacaggccaagacaacgggtaccggtagcggacggtcacacgtggaagacaagtctgtcccatcgatagcgcagcgtaaggatttgtccgtcat

SEQ ID NO: 3 - DNA, 4758 bp, Nannochloropsis
tgatgcgcagtatcaggttgtagggcttctgtttaggatggggtagcggaggaggggatcgtgcgtgaagggcgctgttttcagaggcggaggcggtggctacacgcccgtctgaagcacaagcgaagcactgctgcgccgggccgcgcgtgggcatatttttatgtcagacgcaaggtctctaaaagtgagaaagggccgtcattaagtaagggcgcaagatcgggcctagggcacatactctcccgatatgtgtaaatggccgcttttgtgcctccagcacgcgtgcaaagtgtcaccaaagcgcacgacgcaatggcagtagactccagtgaaatgggtaaaagcggcatgtcattaggtgctttggaaaaaacaatgccaagctttcataaagaagcaaacaagcaagacaaatcataatacccccactcaagagtaactggagatgaccacgttggctaagtccggttgaaaatcggcgggttttcaatttatgtcgacatccctgccccggttcgcgggtcaccatggaaacatactttttttgttgcgccacttctcggccgtaccccacaagacgtatccacgattgcgaaaggtaaagttctatttcatgtcccctggtcgactggtttgtgtaactccttgccaaaaacctacctgttactttatcaccgacggcgttggcacaacaggcactgcaccaaaaaaccgccgcctcgattgggggccgcgaacatgacgtgtcggaacaggaaaagcaaccgctgcaaggcgccggatcttagcgcgcagtcgtagcagcaggaatcgacaatatgtgttctcgtttctgcatacgtcgactctggacagctatgactgacccgactctgaccacacttcatgacccacccaccacacaacgagggcaggtgccgcagcagctttagcacgttgctcctctacatggcctttaaactctcaccacaggtgcctacggcgcctgagccggtccgtgccaccagtacagacgcgcgagacgatgacacggcagacaagtgggttcagcgcctgccaggcatgatccgcctgacgggtcgccacccgtttaacgccgagccgcacaccaaggagctggtcgacgctggcttcattacccccgccgccatgcactatgtgcggtaagtccttcttttctgggtcggccaatccagttcgtgtctctcatcatctttctaaactacaccatcacctacagcaaccacggcccagtacccaagcttgcctgggatgaccaccgcataaccgtgaccggccttggcgtagctgagccccaggttttgtcaatggacgaactggtcgccctgcccaatcgaaccctgcccgtcactcttgtctgcgccggcaatcgccgcaaggaggttaacgtcacccggcagagcaagggcttcagctggggctccggtgcagtgagcacctccatttggacgggcgtgcccctgcacgtgcttctgcgccactgtggcgttgaccccgatgcgttagagcccggacaatactgggtcaacttcgacgggcctgacggggagctgcccaagggcatttatggcacgagtatccccctcctcaaggtaagcattcgggcatatatttatgcatgcttgtgctcattgtcatccagttatgacaaaactatccatcttttcttttttccaggcgctggatccagcacaggatgtgcttgtggccttcaagcagaaccacgagcgcctcctccccgaccacggcttccctgtccgcctcattattccaggtacacaatcaaacatacacatacacacccatacacgcacatacatacatgaaccacagccatattactcactttcctttttatctcctctacttgcagggtacatagggggacggatgatcaaatggcttactcgcatcactatcagccgccaagagtcgcagtccttttaccacttccacgacaatcgcgtcctgccctcgtcggtggaccaagagcgcgcggataatgagggctggtggcgcaagcctgagtacatcatcaacgacctcaaccttaactcggccatcacccatccggtacgtactcgtgtccgcgggcctcagctgggattacgaaagtttacaaacgtgcaagctcgccgcttatactgctgttcgtcattttccctgccagactcacgacgaggagatcccgctgaagaaaggcacttacaaactccaaggctacgcctactgtggtggcggccggcaagtgcaacgcatggaggtctccctcgacgatggcaaggtaggaagtatcaccttgtcgcggttttcacactgctatcattgactcaatcgacatttacacacccattccgactacgcaccacgagtagagctgggaactagctcagctgagcagtgaggagtacccaactgaacacggccgcttctggtgctggcgtatttggaatcttgatgtcgacatcctacgtctggtgagtgcagcttgagaacgagcgagaaacgtctgtgttccatcacgctctcatatatacaatccctccctctctcataggtgggctgtacgaatgtcgcctgtcgtgcatgggacaactcgcagaacacacagcctcgagacttgacgtggaacgtcctaggcagtgagtatttcgtttcctctcttctagagatacttttattccatccatctctatctttacctgtgtctatgagagtaagagtaatcgtcacaccttattcatacccctgaactcccctttccacccctcccttccctacccacagtgatgaacaacagctggttccggcttacaaccgcggttagtctcaacgatcgccagcagcctgtcgtccgcatcaagcacccggcgcccattgctcctggtgggtggatggaggcaggggccgacgagactgtaaatgtacaggccaagacaacgggtaccggtagcggacggtcacacgtggaagacaagtctgtcccatcgatagcgcagcgtaaggatttgtccgtcatcacgcgcgaagagttggcgcggcacaacagcaagtacgtgcggggggaagagggcatgaggaaggtgggtggagggagggcaacgacgatgtttgtccatcaatacgtgtatatgacgcacagtcaaccgctgactaaacctactgcacgaaaatcacagaactgactgctggatcgctgtcaagggtcaggtctacgatgtgaccccctacttgcaggagcacccgggcggcgtggccgccatcgtcatgaacgccggcaaagacgccaccgaggattttgaggtacgcaacatatgaatatgcaagcccgccccgcatgcatcgatgagagcacggcacttaattgtgcccaccaaccacatacacacgcaggcgatccactccaaaagggcctgggctatgctggatgagtatctggtcggcaccctcggggcttctttgacctcctcctcccctgaagcctccgccatcgccgcgcccaaggaggctgccgtggcgctgcaaggcaagaaccgcgccatcaagtgcaagctcgtgttcaaggagtacgagagtcccgacgtcctgcgtatccgatttggcctcccgcagccagaccagcccctgggcctccctgttgggatgcatatcggcctgcgcgccgtgatcaacggtgagagtaccaagcggcaatacacgcccgtgtcggacggggacgccaagggtcacgtggagctgctggtcaaggtacgtgcgtgcaggcaaatgggttttgagtgattggacgatggagcctctctcatcctctgcgcgagctaggaccatctgacattgccagtccccgtcgccccttgcggacctgtagaccccgtagccccccttgcacgcacagcgccttatttcttgacacacatgcacaccctaccacacaggtctaccgcgccaaccagcacccgcgctttcccgacggcgggcttatgtcgcagcacctagaccgtatgtccctcggcgactgcatagatattgacggacccctcggtcacattacttacgagggccccggctgcattcgccaactgggggaggacgtgcatgtcaagcactttgtggcggtcgccggcggcacgggcatcacgccagtcgtgcaggtacgttgacaatcgacgtcataaattttgaggaaaaaaggtgttggtgtcgggaatacgtcgctagtgactgattcgaatccactcaatcaactctcaccgcaggtgcttcgtgccgtgttagagaatccttgcgacactacccgcttttccctcatctatgcggcccgggttccagaggatttgctcctgcgcgaggagctggacgcctgggcggagcagtacgaacagtttacggtaaggaatagtgttctcgacatttggtctcagcttccgctcattttcttcttgacacgactcactaactcaacattctgctttacttatctcttaatttaaggtgcactacaccgtcgatgttcctccccctgattggccgtactccgtcggtttcctcacggccgagatgctggcggcgaatttccccgaggccaccaaggacatgggcgcgcttatctgcggcccgccgccgatggtgaacttcgccgtgaaacccaacctagaaaagcttggttacaccgaggaccagtttttcatcttttaaatgaatgtgggtgcatgcgtgtatgcgtgcatgtatgcgtgt

SEQ ID NO: 4 - DNA, 4020 bp, Nannochloropsis
tgacatggaccagcggcttaagtacgccggtattttccacaggcgcaagcgcacccctgggcgcttcatgatgcggtatgtgttccctctatgcacagctgagtgaaagaaagaaggatgaaaggagacaaggattgagtgcgaataacaatgctctctctgacggatttgtggcatccgaatctcatctacacaccctatcccactcacacacccacacgccaccagaatgcgcattcctaacgggctactcacatccaagcagctcttcttcatctcggaggccatggccaagtacggggacgattatgaggcggtagttgacatcacaacacggcaaaaccttcagcttcgggggctcaaggtaagacatagacgaagaggcggtagaaaagcaggaaggagaagatcatggagataaggaccaaaaaagggttgaaatttattattttccacaccgtccacacgcacagctggaggacacaagcgacctgatcacaggcctggtagagctcggcttgggttcctacagcagtggcctcgacaacgtgcgcaacgttgtgggtagccccctggccggtattgactcgctggaggactttgacacgcgccagatatggtaggtgtgctttggtagtgttggtgtttgttggtgtgaagttctcttttttggagcctctccatgagtggtatgtacggtggggtggctgccgccgtccggcaacggatgagtgacgtgccccatgcataatcttatcaatcaccaggactaaccacacattcaaataccaacacacacaaccctcccccagtaaggagattaacgatatggtcaccggtaacaacaagggaaacccggaatgggccaacttaccccgtaaatttaacattgccgtgtcgggctcccgtgatgactatgcacacacgcacatcaacgatattgggctacaggcggtgcgtcacgagaagacaggtgcgtgtgtgtgagtgtgagatgctatggcctaggtaggcatcggtgggtatgagatatatgcaagtatatatgtatatatgtgagtacagcgagagatagtagaaagataggtaggtctatagatacatacttattcttgtctagttacagaagctttctgtatccgtatctgaatgagcacctgtggatgcaataagtacgaacatggaaggcgcgcgctcactctcacatacccattaacgcccatttcaaaaccccgaacccaggcgaggctggcttcaacgttgtcctcggcggctatttctcgacaaaacgtgctgccgggagcgtggatctaaacctctgggtacctcctggccaggtgctcaatctgtgctattccatccttcgggtaagaccattctttgtgtataagtatttatgagtgcatatgtttgttctttcgctggattatattttcacattgtaatgtattgacaggttaacttgtattgcttttaaagcttgatttttcagcgttcaaatcctgccttatccatacatgatttttcaatgaaaatattcctccgcaattcctctagatcttccgcgaccatggccatcgcaaagaccgtcaaaaagcacgcctgctttggctcatcgaagagtgggggttggagaccttccgcaacgctgtgcttgaagaaatgcagaccaatgccaaatacggcgtgggagaggaaaattcactgccggtcgacggggaacaacgccatgctggtacgatgaactttcctatatttatggtccatcatctaagcttgcttaaacccatcactcctgcatctcttcctataattcgcctttgctcaatcgagggcccgacaaaaagtcagtcaattagactctaacttcctatgtacactcacacgcacacgcacacagaaccatctttcgaacgccgggattacttgggtgtacaccgccaaaaacaaacaggctactcttgggttgggctccatgtccccgtcggtcgtttccgcgccttcgaaattcacgagctgggcagattagcggagaaatacagcgggggggagatgcgattaacggtggaacagaatgtgatctttcccaatgtgcgcgacgaggaggtggcgtctttactggcggatccctttattgctggggggcgatttaagattaatccaggtacataatatatgagaacgggaatgcgttcatttatttggtaaaatctgatagatttagtaatatctgcacatataccgtcctgatggttataatcatttatccgtcttatctgtctgtgagtgaactagactcatatggatcatgtcgggccaacctcttcttacacccatttcccctcccattaccattattatcgaaaacgcgcaataggaccgatcgcccgcgggctggtggcctgcaccggtgcgcaattctgcggtgtcgggcttgtggagacgaagacccgagccattgagctagccgagaagctggaggccgcatatgacattcctcagacagtgcgcatgcacttctccgggtgccctaacagctgcggacagagtcaggtggcggacattggcttgatgggcgcgccggccaaaaaggatggaaaggccgtagaaggcgtggatgtcttcctgggcggtgctgtaggagaaggtgggtgggtgttaggtgttaatatacactactttatgggaggaagggtgtttctttgtgtgtacgcatgagtactgaacatcccactcctgctcgcttgcctgatgtccctaccttttcttcatattttttttgggacataaatctccgcaccctcattcactctgaactcttttctcacacccttctccaaatccaaacccacctaccacagaggcggcgctgggcgagaaattcctaaaaaacgtcgccatgggggaggaagactctgacgttcttagcgcgttgggcgagctcttggttgagcgcttcggggcggtgaagaagtagagaagcgacactggccgtcttgtgtggcactaagttgtgtatgtaaatgaattttgattatttgagaagaatgcggaagggcaggagttgtattcagcagaccgtgttttgattctgaatggagtgaaaagagataggctcaaggtaaaatatgacacgaggaagagtagtggacagatagagaaaaattagcgaatcacgcaaaggaagagcgatgaataaacagacttggaaaaaaaaacaagaatatctggtacaaaggaagagacaatgaaaacgaaaatttaaaacaagaacgttacgtcatcgccggcacctcaaagtatctatcttcccttctttctatcttttccaattttctcttgctgtacactcacacccgtcatttgtacataggcatgcacttatcttccttgccttctcatacatcccgcctccctccctccctcctctcaattcacagtcagttcattctcccccagatctctgaaaaatacacctacctcttcctcagttacctcctccaccactctcttcctccatgccggcttcgctcccttctcgaccaacagtgctctgaccccctcgcagaaatccgactcttgattcatatgtcccattgccacgcgatactccattggaaacgtagccactctcccttcctcccttcccttcctctctccctcttccaatagcctccaagtgactctcaatgccgttggggaggccttgcgaaggagggccaaggtcttctccgcccactccttccttccctccctccctccctcctcccctgcctccaccatagcttccaaccgcctgaacaccccttccaaccctccctcctctccaaacacttcctcgatctcctccgccctcttcgccagcatacatcccctcaccttctgcctcgcttcctcccccccaacggcccatttggcaagcacgccctctacctcctccctccccaccctgccctgcaaccctccctctctctctccctccctcaatccctccacttctctcaccatctcttccaccgt

SEQ ID NO: 5 - DNA, 621 bp, Nannochloropsis
gctgatcttgtccatctcgtgtgccacgggtggcaagaaaagctgggggaaaagacaagatcaacacggcaaagagaatcaagttctctttgtgacgtcttttgggcggtttgacgtgtcgaactcttctttttcttcaattaatcctcacacttttgttttctccatcacatgtagtgaaagaggtacacatgagaactaacggattcgtgatttaaacaaactttttcaaaaacaacacacaagcctttcccccggctccactctcaatgctcaacagatctgtttctcttctattcgccctcctttcacacgcttgcgttcgttgttcttgttttcccctctctaccttcctctcactataaacaaagaaaattttatgtaaaataagggtgacaaaagaagaaccagggagaaaagaaaatgacgggggtaggaaaggactacagagaaaaacatgatgcaggaattcaacactctcataccaagcaatcagcacaaacaaacgaagacagctacgggagaaaggccttatttctcttccggtaggttaagaagggatggacaatctctcgcgccaacactgagtgctgcggctgctactgctgctgctactgctac